(19) United States
(12) Patent Application Publication
(10) Pub. No.: US 2013/0309199 A1
(43) Pub. Date: Nov. 21, 2013

(54) Novel Treatment of Multiple Sclerosis (MS)

(71) Applicants: Irmgard Tegeder, Frankfurt (DE); Gerd Geisslinger, Bad Soden (DE)

(72) Inventors: Irmgard Tegeder, Frankfurt (DE); Gerd Geisslinger, Bad Soden (DE)

(21) Appl. No.: 13/881,088

(22) PCT Filed: Nov. 3, 2011
(86) PCT No.: PCT/EP2011/069319

§ 371 Date: Jul. 11, 2013

(30) Foreign Application Priority Data
Nov. 3, 2010 (GB) 1018519.7

(51) Int. Cl. A61K 31/192; A61K 45/06

(52) U.S. Cl. CPC A61K 31/192; A61K 45/06
USPC 424/85.6; 514/570; 514/1.1; 514/653; 424/133.1; 514/46; 514/626; 514/352

(57) ABSTRACT

The present invention relates to the use of an R-enantiomer of a compound according to the following formula (I) (I), wherein R₁ or R₂ is a group selected from H, —CH₃, —CH₂CH₃, —CH₂CH₂CH₃, and —CH₂CH₂CH₂CH₃ or can be taken together with another to give a cyclopropyl ring, a cyclobutyl ring, a cyclopentyl ring, or a cyclohexyl ring, R₃ is a group selected from —COOH, —COOR₆, —CONH₂, —CONHR₆, —CONR₆R₇, —CONHSO₂R₆, —COO—(CH₂)₃—CH₂OH, —COO—(CH₂)₄—ONO₂, —COO—PhOCH₃—C₂H₂—COO—(CH₂)₄—ONO₂, tetrazolyl, and a —COOH bioisostere, R₄ or R₅ is a group selected from —Cl, —F, —Br, —I, —CF₃, —OCF₃, —SCF₃, —OCH₃, —OCH₂CH₃, —CN, —CH═CH₂, —CH₂OH, and —NO₂, R₆ of R₇ is a group selected from —CH₃, —CH₂CH₃, —CH₂CH₂CH₃, and —CH₂CH₂CH₂CH₃, and m or n is an integer selected from 0, 1, 2, and 3, or a nitro-variant of said compound, and pharmaceutically acceptable salts of said compound, preferably Tarenflurbil (R-Flurbiprofen), for use in the treatment of multiple sclerosis (MS).

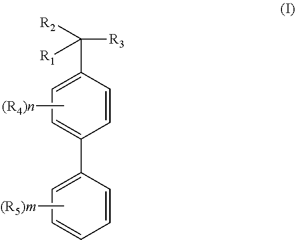

The present invention relates to the use of an R-enantiomer of a compound according to the following formula (I)

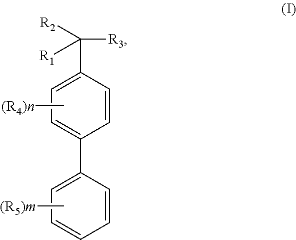

wherein R₁ or R₂ is a group selected from H, —CH₃, —CH₂CH₃, —CH₂CH₂CH₃, and —CH₂CH₂CH₂CH₃ or can be taken together with another to give a cyclopropyl ring, a cyclobutyl ring, a cyclopentyl ring, or a cyclohexyl ring, R₃ is a group selected from —COOH, —COOR₆, —CONH₂, —CONHR₆, —CONR₆R₇, —CONHSO₂R₆, —COO—(CH₂)₃—CH₂OH, —COO—(CH₂)₄—ONO₂, —COO—PhOCH₃—C₂H₂—COO—(CH₂)₄—ONO₂, tetrazolyl, and a —COOH bioisostere, R₄ or R₅ is a group selected from —Cl, —F, —Br, —I, —CF₃, —OCF₃, —SCF₃, —OCH₃, —OCH₂CH₃, —CN, —CH═CH₂, —CH₂OH, and —NO₂, R₆ of R₇ is a group selected from —CH₃, —CH₂CH₃, —CH₂CH₂CH₃, and —CH₂CH₂CH₂CH₃, and m or n is an integer selected from 0, 1, 2, and 3, or a nitro-variant of said compound, and pharmaceutically acceptable salts of said compound, preferably Tarenflurbil (R-Flurbiprofen), for use in the treatment of multiple sclerosis (MS).

MS is a chronic inflammatory demyelinating disease which affects the central nervous system (CNS). The most common initial course of the disease is the relapsing-remitting subtype, which is characterized by unpredictable attacks (relapses) followed by periods of relative remission with no new signs of disease activity.

Different therapies are used for patients experiencing acute attacks, for patients who have the relapsing-remitting subtype, for patients who have the progressive subtypes, and for managing the various consequences of MS. The primary aims of therapy are returning function after an attack, preventing new attacks, and preventing disability.

The following therapies can be used in case of multiple sclerosis: interferon beta 1a and 1b, Glatiramer, mitoxantron, Natalizumab (a monoclonal antibody against intergrin alpha4beta1) and glucocorticoids for the treatment of acute attacks. FTY-720 (Fingolimod, a sphingosin-1-phosphate analogon, which has been recently approved for medical use), cladribin (immune suppressive), Teriflunomid (immune suppressive) and Fampridin (4-aminopyridine, potassium channel inhibitor) are in clinical development. In addition, the efficiency of HMG-CoA reductase inhibitors and certain cannabinoids was studied experimentally.

Despite many promising results coming from research with the drug Fingolimod and the registration of Natalizumab for a treatment of the relapsing-remitting MS there is no cure, at best a reduction of the frequency of the attacks, and thus the delay of the neurological deficits.

In case of a primary progressive MS, no effective treatment is known. Furthermore, all available drug-based therapies (beta-interferon, Glatiramer, mitoxantron, Natalizumab) lead to substantial toxicities.

Tarenflurbil (R-Flurbiprofen) (chemical name (R)-2-(2-fluoro-4-phenylphenyl)propionic acid) was tested in 2008 as a potential candidate for the treatment of Alzheimer's disease. Nevertheless, the further development for this indication was stopped after an insufficient improvement of cognitive functions was found.

R-Flurbiprofen, together with, for example, Ibuprofen and Naproxen, belongs to the group of 2-aryl propionic acids (profens). Just like Ibuprofen, R-Flurbiprofen is a by-product of the marketed racemic Flurbiprofen, the active agent of which is thought to be the S-enantiomer. Flurbiprofen is currently in clinical trials for the treatment of metastatic prostate cancer.

Cardozo et al. (in: Cardozo L D, Stanton S L, Robinson H, Hole D. Evaluation of flurbiprofen in detrusor instability. Br Med J. 1980 Feb. 2; 280(6210):281-2) describe a double-blind, cross-over trial of the prostaglandin synthetase inhibitor Flurbiprofen and a placebo in case of women with detrusor instability (27 cases idiopathic, and three secondary to multiple sclerosis). Frequency, urgency, and urge incontinence were all significantly reduced with Flurbiprofen (P less than 0.001, P less than 0.025, and P less than 0.025 respectively), as was the detrusor-pressure rise during bladder filling (P less than 0.01). Side effects, however, occurred in 13 patients while taking Flurbiprofen compared with five while taking placebo (P less than 0.025). After the trial 19 patients wished to continue with Flurbiprofen. Flurbiprofen is a useful treatment for idiopathic detrusor instability and is well tolerated by most patients.

US 2009-0162421 describes the use of tarenflurbil and/or a pharmaceutically tolerable salt or derivative thereof in enantiomerically-pure for the production of a drug for the treatment of pain-associated neuropathy. In contrast to US 2009-0162421, the present invention relates to the use of R-Flurbiprofen for the treatment of the neuroimmunological pathology of multiple sclerosis and thereby for the prevention or progression of a loss of motor functions and neurodegeneration which results from immune-mediated demyelination. Prevention of demyelination which is due to an autoimmune attack of the myelinating cells in the central nervous system may be associated with in a reduction of other neurological MS-associated symptoms which may include a centrally mediated hypersensitivity of the nociceptive system with neuropathic pain-like phenomena. However, neuropathic pain caused by nerve injury (traumatic, inflammatory, metabolic, ischemic, toxic etc.) and described in US 2009-0162421 is primarily caused by hyperexcitability of injured or secondary nociceptive neurons. Some forms are associated with a microglia activation in rodent models which however, strongly differs from the general and widespread immune activation in Multiple Sclerosis, both in terms of mechanisms and localization.

Furthermore, neuropathic pain is not an autoimmune disease and the efficacy and use of R-Flurbiprofen described in US 2009-0162421 for the treatment of neuropathic pain is mediated through different mechanisms compared to the here described immunmodulatory features. Reduction of neuropathic pain with R-Flurbiprofen mostly results from a prevention of the maladaptive neuronal changes which occur after axonal injury whereas its efficacy in Multiple Sclerosis mostly results from immunmodulatory effects on T-cells preventing thereby the autoimmune mediated destruction of the myelin sheaths of the neurons, i.e. a destruction of oligodendrocytes and Schwann cells. If this myelin destruction occurs in the thalamocortical tract, i.e. the ascending pain pathway, it may cause symptoms of central neuropathic pain in MS. Means, secondarily R-Flurbiprofen may reduce this form of neuropathic pain or the progression thereof in MS patients. In summary, an efficacy of R-Flurbiprofen in neuropathic pain (e.g. mostly caused by trauma, Zoster infection, diabetes or ischemia) however, did not allow for the prediction of its immunmodulatory effects and the here described prevention of immune-mediated myelin destruction and motor function loss in MS.

Hence, the present invention is based on a novel use of R-Flurbiprofen for immune modulation and motor function preservation in multiple sclerosis which is an autoimmune disease of the nervous system, whereas relates to the use of R-Flurbiprofen for the prevention or reversal of nociceptive neuron hyperexcitability caused most frequently by peripheral nerve trauma, inflammation, metabolic dysfunctions or ischemia.

Barkhof et al. (in: Barkhof F, van Waesberghe J H, Uitdehaag B M, Polman C H. Ibuprofen does not suppress active multiple sclerosis lesions on gadolinium-enhanced MR images. Ann Neurol. 1997 December; 42(6):982) examine the effect of Ibuprofen on the number and size of MS-lesions in the MRT. The authors describe that the effect of co-administered beta-interferons is not essentially influenced by the co-medication.

In view of the above, an ongoing demand exists for the development of new and effective treatments for MS, in particular for the primary progressive MS.

In a first aspect of the present invention, this object of the present invention is solved by an R-enantiomer of a compound according to the following formula (I)

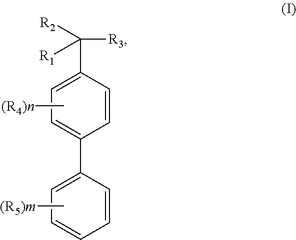

wherein R₁ or R₂ is a group selected from H, —CH₃, —CH₂CH₃, —CH₂CH₂CH₃, and —CH₂CH₂CH₂CH₃ or can be taken together with another to give a cyclopropyl ring, a cyclobutyl ring, a cyclopentyl ring, or a cyclohexyl ring, R₃ is a group selected from —COOH, —COOR₆, —CONH₂, —CONHR₆, —CONR₆R₇, —CONHSO₂R₆, —COO—(CH₂)₃—CH₂OH, —COO—(CH₂)₄—ONO₂, —COO—PhOCH₃—C₂H₂—COO—(CH₂)₄—ONO₂, tetrazolyl, and a —COOH bioisostere, R₄ or R₅ is a group selected from —Cl, —F, —Br, —I, —CF₃, —OCF₃, —SCF₃, —OCH₃, —OCH₂CH₃, —CN, —CH═CH₂, —CH₂OH, and —NO₂, R₆ of R₇ is a group selected from —CH₃, —CH₂CH₃, —CH₂CH₂CH₃, and —CH₂CH₂CH₂CH₃, and m or n is an integer selected from 0, 1, 2, and 3, or a nitro-variant of said compound, such as (R)-2-(2-fluoro-biphenyl-4-yl)propionic acid 4-nitrooxybutyl ester, and pharmaceutically acceptable salts of said compound for use in the treatment of multiple sclerosis (MS).

Preferably, R₁ is selected from H. Further preferred is the use of a compound according to the present invention which is selected from the group of
(R)-2-(2-fluoro-4-phenylphenyl)propionic acid,
(R)-2-(2-fluoro-biphenyl-4-yl)propionic acid 4-nitrooxybutyl ester,
(R)-1,1′-biphenyl)-4-acetic acid 2-fluoro-α-methyl-4-hydroxybutylester,
(R)-3-[4-(2-fluoro-α-methyl-[1,1′-biphenyl]-4-acetyloxy)-3-methoxyphenyl]-2-propenoic acid 4-nitrooxybutyl ester,
(R)-2-methyl-2(2-fluoro-4′-trifluoromethylbiphen-4-yl)propionic acid,
(R)-2-methyl-2(2-fluoro-4′cyclohexylbiphen-4-yl)propionic acid,
(R)-2-(2-fluoro-3′,5′-bis(chloro)biphen-4-yl)propionic acid amide,
(R)-2-(2-fluoro-4′-trifluoromethylbiphen-4-yl)propionic acid,
(R)-2-(2-fluoro-3′-trifluoromethylbiphen-4-yl)propionic acid,
(R)-2-(2-fluoro-3′,5′-bis(trifluoromethyl)biphen-4-yl)propionic acid,
(R)-2-(4′-cyclohexyl-2-fluorobiphen-4-yl)propionic acid,
(R)-2-(2-Fluoro-1,1′-biphenyl-4-yl)-2-methylpropanoic acid,
and (R)-5-[1-(2-Fluoro-biphenyl-4-yl)-1-methyl-ethyl]-2H-tetrazole.

Further preferred is R-Flurbiprofen or Nitro-R-Flurbiprofen for use in the treatment of multiple sclerosis (MS). Even further preferred is the use of a compound as above, preferably R-Flurbiprofen or Nitro-R-Flurbiprofen for the production of a medicament for the treatment of multiple sclerosis (MS). Another aspect of the present invention relates to a method for treating of multiple sclerosis (MS), comprising administering to a patient in need thereof a therapeutically effective amount of a compound according to the present invention, and preferably R-Flurbiprofen or Nitro-R-Flurbiprofen. The primary aims of therapy according to the present invention are returning function after an attack, preventing new attacks, and preventing disability. Thus, the present treatments preferably do relate to a treatment that is different from the treatment of pain-associated neuropathy, i.e. preferably does not involve the treatment of pain-associated neuropathy.

In the context of the present invention, treatment shall include both preventive and/or actual treatment of the disease symptoms of MS as described herein, which can be alleviated and/or even completely removed using said treatment.

The present invention is based on the surprising finding that an R-enantiomer of a profen-compound, namely R-Flurbiprofen, reduces/inhibits the occurrence of paralyses in the EAE-model of multiple sclerosis in mice (EAE: experimental autoimmune encephalomyelitis), and nearly completely blocks the MOG-induced activation of microglia and immune cell infiltration in the lumbal spinal cord. Therefore, due to the similarities between the human situation and the mouse model, an at least similar effect of Tarenflurbil (R-Flurbiprofen) in the human patient is expected.

In contrast to its S-isomer, R-Flurbiprofen does not inhibit the cyclooxygenases and has no effect on the prostaglandin synthesis. Even at high daily dosage and long-term therapy, no essential toxicity is known. Another aspect of the present invention thus relates to a method for treating of multiple sclerosis (MS) which is free from inhibiting cyclooxygenases and has no effect on the prostaglandin synthesis, comprising administering to a patient in need thereof a therapeutically effective amount of a compound according to the present invention, and preferably R-Flurbiprofen or Nitro-R-Flurbiprofen. Still another aspect of the present invention relates to an improved method for treating of multiple sclerosis (MS) by preventing and/or reducing the toxicity involved in said treatment resulting from the inhibition of cyclooxygenases, comprising administering to a patient in need thereof a therapeutically effective amount of a compound according to the present invention, and preferably R-Flurbiprofen or Nitro-R-Flurbiprofen.

R-Flurbiprofen was tested as a potential therapy in Alzheimer's disease, but finally did not exhibit any significant effect. Furthermore, Barkhof et al. (see above) excluded an individual effect of Ibuprofen on MS, and thus the person of skill would have expected that Flurbiprofen or a compound as depicted in the above formula (I) would also be ineffective in MS.

Preferably, the MS to be treated is relapsing-remitting or progressive MS.

Even at high daily dosage and long-term therapy, no essential toxicity of R-Flurbiprofen is known. Thus, generally any dosage of a compound according to the present invention, and preferably R-Flurbiprofen or Nitro-R-Flurbiprofen, can be used which exhibits an advantageous effect on the symptoms of the MS to be treated. Respective effective dosages can be readily determined by the person of skill and/or the attending physician. Preferred is the use of a compound according to the present invention, and preferably R-Flurbiprofen or Nitro-R-Flurbiprofen, according to the invention, wherein said compound according to the present invention, and preferably R-Flurbiprofen or Nitro-R-Flurbiprofen, is provided in an amount of between 50 mg to 3000 mg, preferably of between 100 mg to 1500 mg, more preferably between 300 mg to 1200 mg per dosage form. Further preferred is a use, wherein said compound according to the present invention, and preferably R-Flurbiprofen or Nitro-R-Flurbiprofen, is provided in a dosage of between 5 mg/kg of body weight to 15 mg/kg of body weight of the patient to be treated per day.

In general, the a compound according to the present invention, and preferably R-Flurbiprofen or Nitro-R-Flurbiprofen, can be provided to the patient in any suitable and effective manner, such as orally, rectally or by injection. Preferred is orally. Furthermore, the compound according to the present invention, and preferably R-Flurbiprofen or Nitro-R-Flurbiprofen, can be provided to the patient in any suitable and effective pharmaceutically acceptable form, such as in the form of a tablet, capsule, dragée, powder, suppository, gel and/or as solution for injection.

The compound according to the present invention, and preferably R-Flurbiprofen or Nitro-R-Flurbiprofen, can be used alone or in combination with other compounds and treatments that are available for the therapy and/or treatment of MS. Preferably, a neurological symptomatic is effected. The combination includes a simultaneous or spaced apart use of the compounds and treatments. The combination also includes any synergistic effect of the compounds and treatments that are available for the therapy and/or treatment of MS. Therefore, another aspect of the present invention is the use of a compound according to the present invention, and preferably R-Flurbiprofen or Nitro-R-Flurbiprofen, wherein said a compound according to the present invention, and preferably R-Flurbiprofen or Nitro-R-Flurbiprofen, is provided in combination with at least one additional therapeutic against MS, such as, for example, interferon beta 1a or 1b, Glatiramer, mitoxantron, Natalizumab, glucocorticoid, Fingolimod, cladribin, Teriflunomid, Fampridin, a HMG-CoA reductase inhibitor or a cannabinoid. Preferred is interferon beta 1a or 1b.

Another aspect of the present invention then relates to a method for treating, and in particular reducing, the symptoms of multiple sclerosis (MS), preferably the neurological deficits, comprising administering to a patient in need thereof a therapeutically effective amount of a compound according to the present invention, and preferably R-Flurbiprofen or Nitro-R-Flurbiprofen, as described herein.

Yet another aspect of the present invention then relates to a method for reducing the frequency, occurrence, and/or severity of attacks in multiple sclerosis, such as relapsing-remitting MS, comprising administering to a patient in need thereof a therapeutically effective amount of a compound according to the present invention, and preferably R-Flurbiprofen or Nitro-R-Flurbiprofen, as described herein.

In the EAE model of multiple sclerosis, mice that were treated with R-Flurbiprofen (9 mg/d in drinking water), did not exhibit paralyses, whereas at the same time control animals that were treated with placebo developed a para- to tetraparesis and had to be sacrificed in accordance with the termination criteria. In animals treated with R-Flurbiprofen, nearly no activation of the microglia or T-cell infiltration of the white matter was detectable in the distal spinal cord, i.e. the main localization of the pathological immunological manifestations of EAE. The T-cell mediated destruction of the myelin sheaths and direct damage of axons is the cause for the neurological deficits.

The biological mechanisms of the effect of R-Flurbiprofen in EAE are only partially known. R-Flurbiprofen leads to a complex modulation of lipid-signal molecules and modulation of transcription factors (such as, for example, NF-kappaB and PPAR), and therefore to an immune modulation and change of the neuroimmulogical communication. Therefore, the moderate modulating effects explain the advantageous effect/toxicity profile of the compound.

As mentioned above, with currently available medications no cure of MS, but only a reduction of the attacks and inhibition of progression can be achieved. In some cases with primary progressive (non-relapsing remitting) MS no effective therapy is available. The disease leads to increasing paralyses and is finally lethal. In addition, the patient suffer from pain because of spasticities and neuroimmunological damage in pain-conducting neurons or centers.

The main advantage of a compound according to the present invention, and preferably R-Flurbiprofen or Nitro-R-Flurbiprofen, is its low toxicity, which is sufficiently proven for R-Flurbiprofen. Even with a long-term therapy in old patients no significant side-effects occurred. Usually, mild side-effects do not lead to a termination of the therapy.

The reduction of the neurological symptoms and the neuro-immunological pathology as found in animals treated with R-Flurbiprofen was substantial. Therefore, it is assumed that a compound according to the present invention, and preferably R-Flurbiprofen or Nitro-R-Flurbiprofen, will also reduce the symptomatic in patients with MS.

The present invention will now be explained in the following examples with reference to the accompanying figures, without being limited thereto. For the purposes of the present invention, all references as cited herein are incorporated by reference in their entireties. In the Figures,

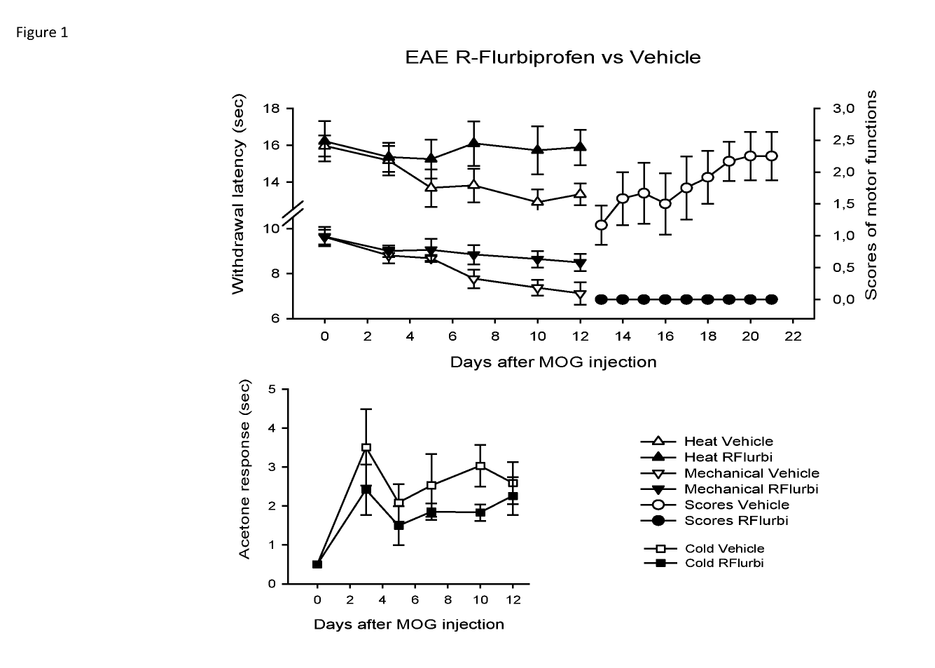

FIG. 1 shows the effect of R-Flurbiprofen (9 mg/kg/d p.o.) in the EAE-model of multiple sclerosis in C57BL6/J mice. An experimental autoimmune encephalomyelitis (EAE) was induced by subcutaneous injection of 100 μg MOG35-55 in 200 μl CFA, followed by an intraperitoneal injection of 200 ng Pertussis-toxin (PTX). The PTX-injection was repeated 2 days later. R-Flurbiprofen was applied p.o. in the drinking water (225 μg/4 ml per day) (n=6). The control group received placebo (n=9). The nociceptive behavior was exclusively studied before manifestation of motor-dysfunctions. Mechanic hyperalgesia, thermal hyperalgesia (top, left y-axis) and cold allodynia (bottom) was measured. Hyperalgesia is an early manifestation of the neuroimmunological activation. The motoric function was detected based on a standard-scoring system: Score 0.5: distal paresis of the tail; score 1: complete paralysis of the tail; Score 1.5: Paresis of the tail and mild paresis of the hind legs; Score 2.0: severe paresis of one hind leg; Score 2.5: medium paraparesis of the hind legs; Score 3.0: complete paralysis of both hind legs; Score 3.5: complete paralysis of both hind legs and paresis of one front leg; Score 4: complete paralysis (tetraplegia), moribund state or death. The animals were sacrificed starting at Score 3.5. All tests were performed by an observer, who was not informed about the treatment. R-Flurbiprofen reduced the hyperalgesia which was detectable at the beginning and completely inhibited the development of motor dysfunctions (P<0.05). Animals that were treated with R-Flurbiprofen, did not show indications of demyelinisation during the observation period. In the placebo group all animals reached scores of 2.5 to 3.5.

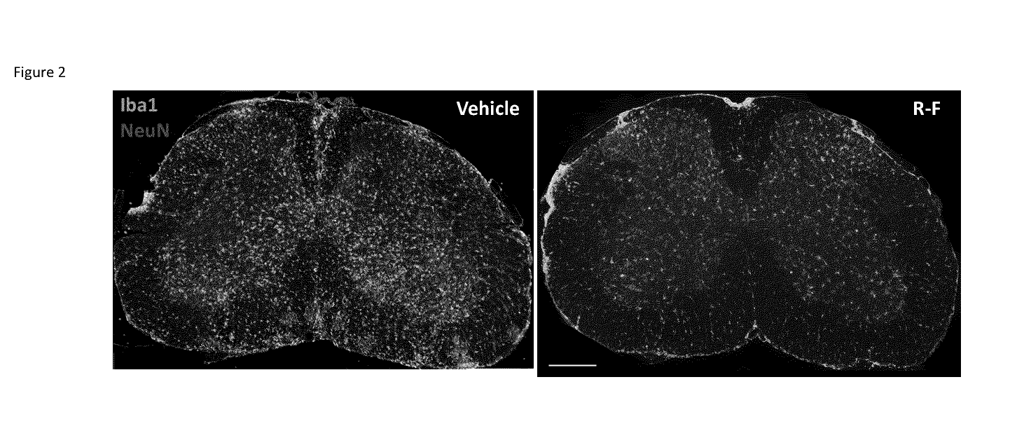

FIG. 2 shows the microglial activation in the spinal cord (lumbal) 3 weeks after injection von MOG35-55 in C57BL/6 mice treated with R-Flurbiprofen (9 mg/kg/d p.o.) or placebo. For the histological examination the mice were intracardially perfused with 4% paraformaldehyde; the tissue was prepared, post-fixed in PFA and protected for cryo-artifacts by preservation in 20% sucrose. The tissue was embedded in OCT and cut on the cryotome (16 μm). An immune-staining was performed using a primary-antibody, which was directed specifically against the microglia-marker Iba-1. Neurons were detected with anti-NeuN. After staining by fluorochrome-labeled secondary antibody, pictures were taken using a Zeiss fluorescence-microscope. In R-Flurbiprofen treated animals no activation of the microglial cells was detectable. That is, the spinal cord did not show any pathological changes and was not different from the healthy control animals. In contrast, EAE animals treated with placebo showed a massive activation and proliferation of the microglia.

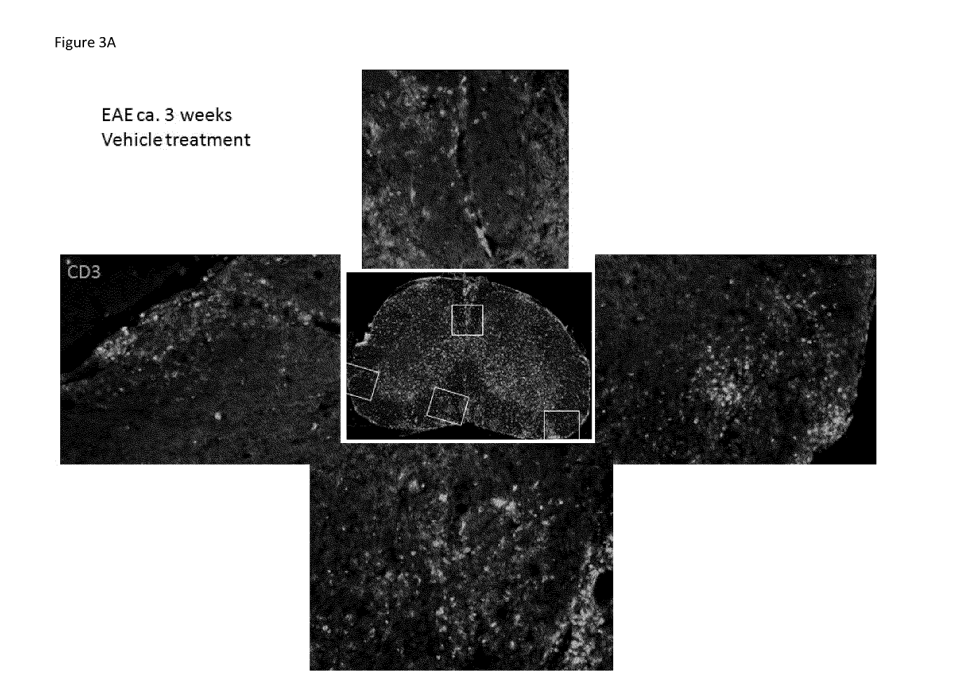

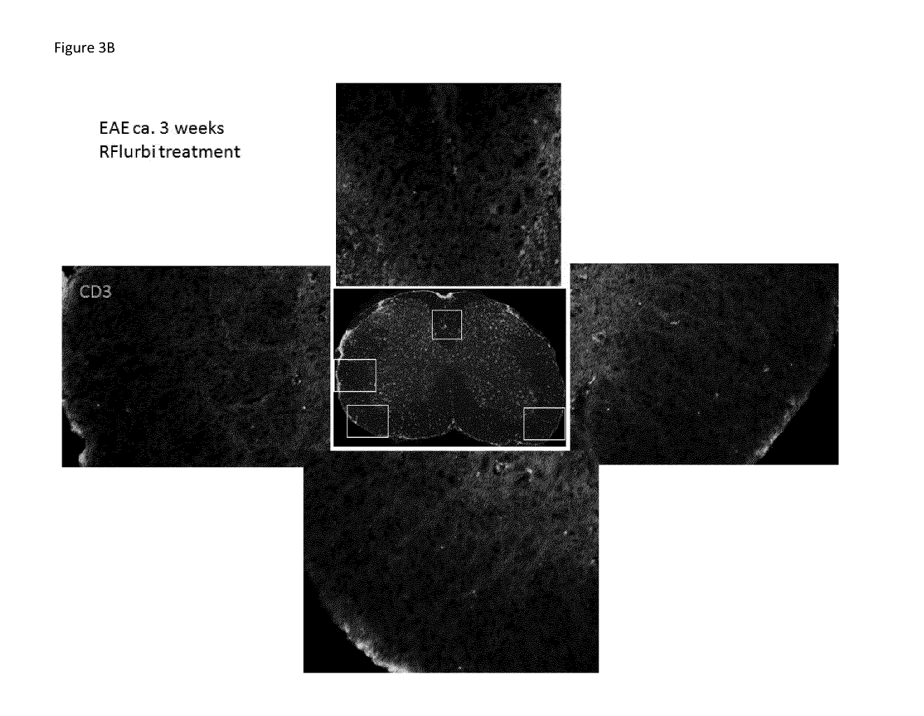

FIG. 3 shows the T-cell infiltration in the dorsal horn of the lumbal spinal cord 3 weeks after injection of MOG35-55 upon treatment with R-Flurbiprofen (9 mg/kg/d p.o.) (FIG. 3A) or placebo (FIG. 3B). The preparation took place as described above. T-cells were detected using an anti-CD3 antibody. The T-cell infiltration into the area of the white substance in the ventral and dorsal horn was nearly completely inhibited by the treatment with R-Flurbiprofen.

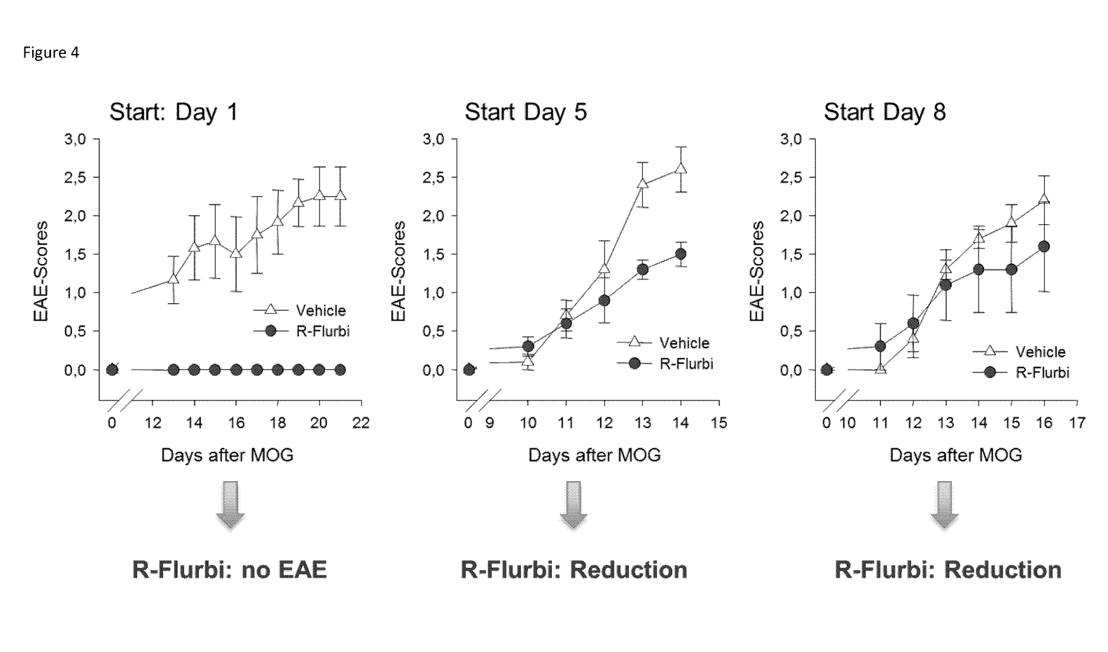

FIG. 4 shows the time course of the clinical scores in the EAE model of multiple sclerosis depending on the start of R-Flurbiprofen treatment. EAE was induced by injection of MOG35-55 peptide and pertussis toxin in C57BL6 mice. Vehicle or R-Flurbiprofen (9 mg/kg/d p.o.) treatment was initiated at the day of immunization (Day 1) or 5 or 8 days after immunization with MOG. Clinical EAE-scores were assessed as described in FIG. 1. R-Flurbiprofen treated animals did not develop EAE when therapy was started on day 1 and had significantly reduced EAE-scores when treatment was started 5 or 8 days after MOG injection. The areas under the scores x time courses were statistically analyzed with t-tests, P<0.05.

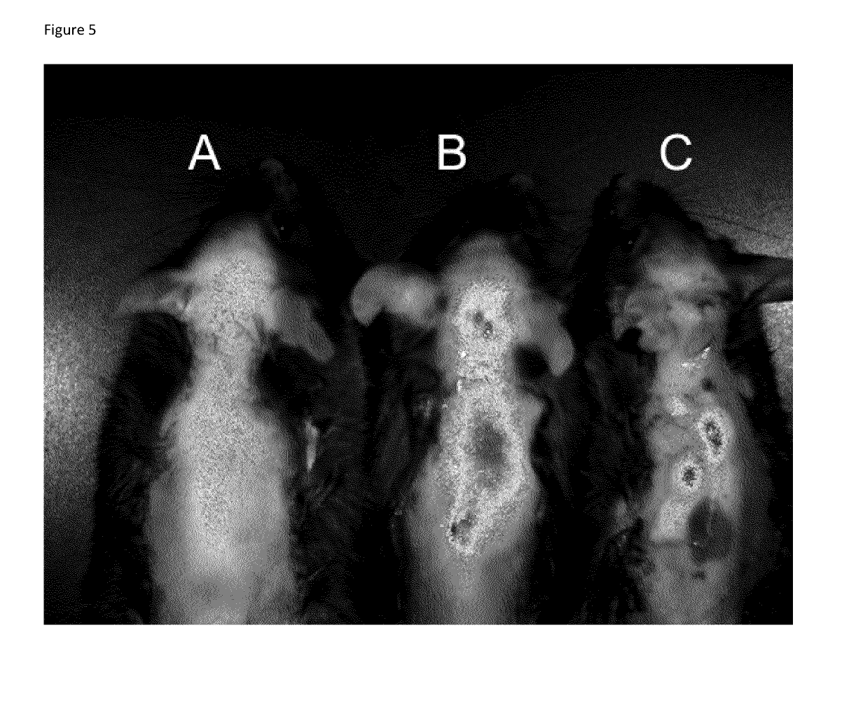

FIG. 5 shows the Imaging of neuroinflammation in the EAE model of multiple sclerosis. Near-infrared Imaging (Maestro-Imaging Platform) was performed 3 days after injection of ProSense 680 i.v. in C57BL6 mice treated with vehicle or R-Flurbiprofen (9 mg/kg/d p.o.) starting 3 days after induction of EAE by injection of MOG35-55 peptide and pertussis toxin. ProSense 680 is a fluorescent substrate of cathepsins and allows for analysis of inflammation. A: Control mouse without ProSense injection. B: Vehicle treatment. C: Treatment with R-Flurbiprofen.

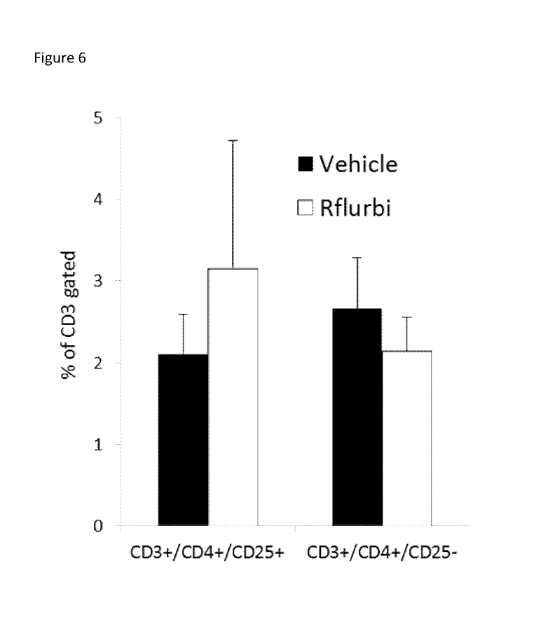

FIG. 6 shows the flow cytometry analysis of CD4+/CD25+ T-cells in the spinal cord in the EAE model of multiple sclerosis. C57BL6 mice were treated with vehicle or R-Flurbiprofen (9 mg/kg/d p.o.) starting 3 days after induction of EAE by injection of MOG35-55 peptide and pertussis toxin. Single cell suspensions were prepared from the lumbar spinal cord segment when animals reached a clinical score of 1.5-2. Analysis of T-cells was performed on a Flow Cytometer (BD FACS Conto II) with specific antibodies directed against cell surface marker proteins. T-cells were identified by CD3 and subsequently gated for T-cell subtypes according to expression of CD markers. R-Flurbiprofen treated animals showed a higher number of CD4+/CD25+ T-cells suggesting a higher number of regulatory T-cells which have protective functions in EAE.

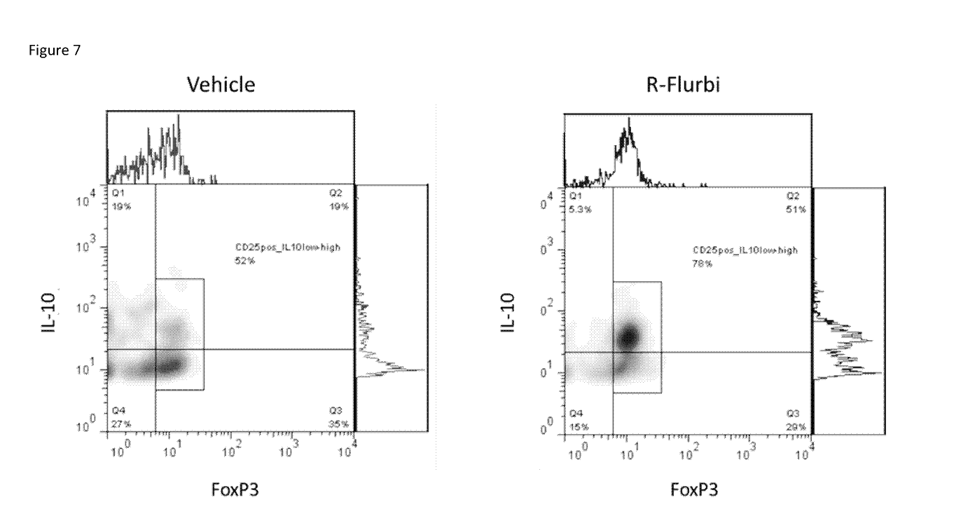

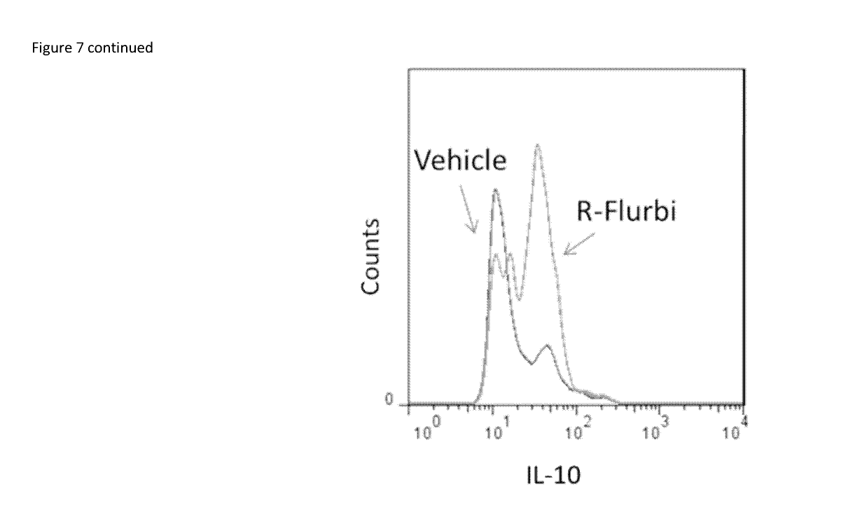

FIG. 7 shows the flow cytometry analysis of IL10+ and FoxP3+ T-cells in the EAE model of multiple sclerosis. C57BL6 mice were treated with vehicle or R-Flurbiprofen (9 mg/kg/d p.o.) starting 3 days after induction of EAE by injection of MOG35-55 peptide and pertussis toxin. Single cells suspensions were prepared from the spleen when animals reached a clinical score of 1.5-2. Analysis of T-cells was performed on a Flow Cytometer (BD FACS Conto II) with specific antibodies directed against cell surface marker proteins. T-cells were identified by CD3 and subsequently gated for T-cell subtypes. For analysis of intracellular cytokines splenocytes from EAE mice treated with vehicle or R-Flurbiprofen were stimulated with 50 ng/ml PMA+500 ng/ml ionomycin for 2 h at 37° C., followed by brefeldin A 10 μg/ml for 2 h, at 37° C. to prevent the release of the cytokines. R-Flurbiprofen treated mice showed a higher fraction of CD4+/CD25+/FoxP3+ regulatory T-cells and an increase of anti-inflammatory IL-10 production.

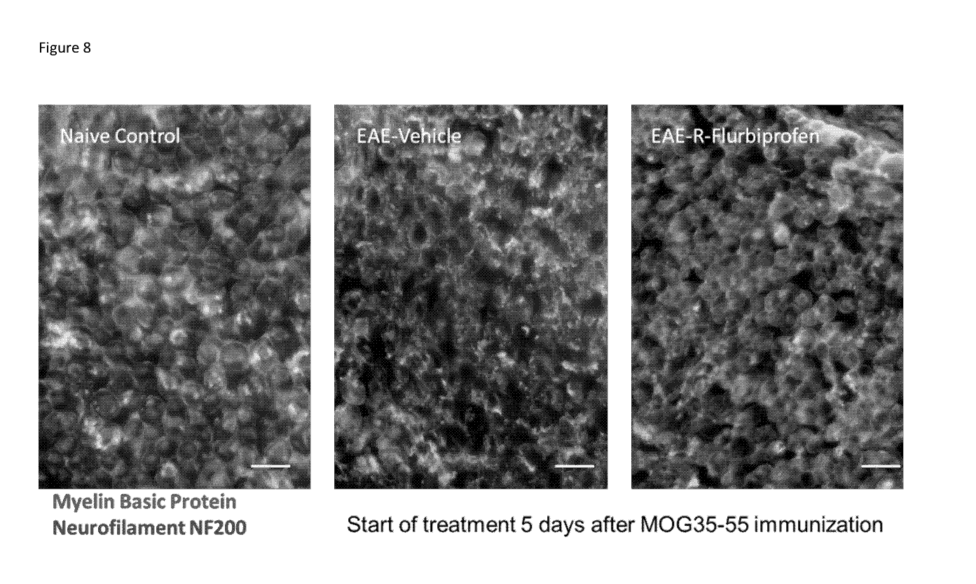

FIG. 8 shows the immunofluorescent analysis of demyelination in the optical nerve in the EAE model of Multiple Sclerosis. C57BL6 mice were treated with vehicle or R-Flurbiprofen (9 mg/kg/d p.o.) starting 5 days after induction of EAE by injection of MOG35-55 peptide and pertussis toxin. Mice were intracardially perfused with phosphate buffered saline followed by PFA 4% fixation when animals reached a clinical score of 1.5-2. Optical nerves were removed, postfixed, overnight cryoprotected in 20% sucrose and cut on a cryotome. Sections were incubated with an antibody directed against myelin basic protein and counter-stained with the neuronal marker antibody NeuN. Analysis was done with a fluorescent microscope (Zeiss Axiovert). The images show that R-Flurbiprofen treatment substantially reduces the destruction of the myelin sheaths surrounding the neuronal fibers as compared to vehicle treated mice.

1. A method for treating multiple sclerosis (MS) wherein said method comprises administering, to a subject in need of such treatment, an R-enantiomer of a compound according to the following formula (I) 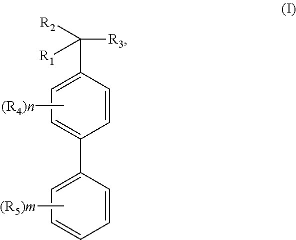
wherein
R₁ and R₂ are each, independently, a group selected from H, —CH₃, —CH₂CH₃, —CH₂CH₂CH₃, and —CH₂CH₂CH₂CH₃ or can be taken together with another group to give a cyclopropyl ring, a cyclobutyl ring, a cyclopentyl ring, or a cyclohexyl ring;
R₃ is a group selected from —COOH, —COOR₆, —CONH₂, —CONHR₆, —CONR₆R₇, —CONHSO₂R₆, —COO—(CH₂)₃—CH₂OH, —COO—(CH₂)₄—ONO₂, —COO—PhOCH₃—C₂H₂—COO—(CH₂)₄—ONO₂, tetrazolyl, and a —COOH bioisostere;
R₄ and R₅ are each, independently, a group selected from —Cl, —F, —Br, —I, —CF₃, —OCF₃, —SCF₃, —OCH₃, —OCH₂CH₃, —CN, —CH═CH₂, —CH₂OH, and —NO₂;
R₆ and R₇ are each, independently, a group selected from —CH₃, —CH₂CH₃, —CH₂CH₂CH₃, and —CH₂CH₂CH₂CH₃;
and m and n are each, independently, an integer selected from 0, 1, 2, and 3;
or a nitro-variant or pharmaceutically acceptable salt of said compound.

2. The method according to claim 1, wherein said R-enantiomer is selected from R-Flurbiprofen (Tarenflurbil) and Nitro-R-Flurbiprofen.

3. The method according to claim 1, wherein said MS is relapsing-remitting or progressive MS.

4. The method according to claim 1, wherein said R-enantiomer is administered in an amount of between 50 mg and 3000 mg.

5. The method according to claim 1, wherein said R-enantiomer is administered to the subject in a dosage of between 5 mg/kg of body weight of the subject and 15 mg/kg of body weight of the subject per day.

6. The method according to claim 1, wherein said R-enantiomer is administered orally, rectally or by injection.

7. The method according to claim 1, wherein said R-enantiomer is provided as a tablet, capsule, dragée, powder, suppository, gel or a solution for injection.

8. The method according to claim 1, wherein said R-enantiomer is provided in combination with at least one additional therapeutic agent against MS.

9. The method according to claim 4, wherein said R-enantiomer is administered in an amount between 10 mg and 1500 mg.

10. The method according to claim 8, wherein said at least one additional therapeutic agent against MS is selected from interferon beta 1a or 1b, Glatiramer, mitoxantron, Natalizumab, glucocorticoid, Fingolimod, cladribin, Teriflunomid, Fampridin, a HMG-CoA reductase inhibitor and cannabinoids.

11. A pharmaceutical composition comprising an R-enantiomer of a compound as set forth in claim 1.

12. The pharmaceutical composition, according to claim 11, further comprising at least one additional therapeutic against MS.

13. The pharmaceutical composition, according to claim 12, wherein said at least one additional therapeutic agent against MS is selected from interferon beta 1a or 1b, Glatiramer, mitoxantron, Natalizumab, glucocorticoid, Fingolimod, cladribin, Teriflunomid, Fampridin, a HMG-CoA reductase inhibitor and cannabinoids.
